(19) United States
(12) Patent Application Publication
(10) Pub. No.: US 2010/0142678 A1
(43) Pub. Date: Jun. 10, 2010

(54) MINIMALLY INVASIVE PARTICLE BEAM CANCER THERAPY APPARATUS

(75) Inventors: Nam Soo Myung, Gyeonggi-do (KR); Nak Woong Eum, Daejeon (KR); Seong Mo Park, Daejeon (KR); Moon Youn Jung, Daejeon (KR); Seon Hee Park, Daejeon (KR)

Correspondence Address: RABIN & Berdo, PC, 1101 14TH STREET, NW, SUITE 500, WASHINGTON, DC, 20005, US

(73) Assignee: ELECTRONICS AND TELECOMMUNICATIONS RESEARCH INSTITUTE, Daejeon (KR)

(21) Appl. No.: 12/632,746

(22) Filed: Dec. 7, 2009

(30) Foreign Application Priority Data
Dec. 8, 2008 (KR) 10-2008-0124009
Apr. 13, 2009 (KR) 10-2009-0031778

(51) Int. Cl. A61N 5/10

(52) U.S. Cl. 378/65; 606/33

(57) ABSTRACT

A minimally invasive particle beam cancer therapy apparatus that can be inserted into the body and deliver a particle beam onto a cancer cell generated in the body. The minimally invasive particle beam cancer therapy apparatus may include: a particle beam delivery system delivering a particle beam onto a diseased part formed inside a therapy subject, the particle beam delivery system being partially inserted into the therapy subject when delivering the particle beam; a medical apparatus body shaped like a pipe having a predetermined length and physically connected to the particle beam delivery system, the medical apparatus being partially inserted into the therapy subject in a longitudinal direction along with the particle beam delivery system being partially inserted into the therapy subject to help the insertion of the particle beam delivery system into the therapy subject; and a control system controlling a driving operation of the particle beam delivery system.

### CROSS-REFERENCE TO RELATED APPLICATIONS

This application claims the priorities of Korean Patent Application Nos. 10-2008-0124009 filed on Dec. 8, 2008, and 10-2009-0031778 filed on Apr. 13, 2009, in the Korean Intellectual Property Office, the disclosures of which are incorporated herein by reference.

### BACKGROUND OF THE INVENTION

1. Field of the Invention

The present invention relates to medical apparatuses, and more particularly, to a minimally invasive particle beam cancer therapy apparatus that can be inserted into the body and deliver a particle beam onto a cancer cell generated in the body.

2. Description of the Related Art

In general, radiation therapy used to treat cancer is a non-invasive therapy by which cancer within the human body is treated from outside the body without surgery and anesthesia, thereby reducing physical tiredness in patients.

However, when disease is treated with radiation using X-rays or Gamma rays, radiation may be slightly absorbed into the body to destroy cells while being delivered to the inside of the body, bodily tissues both anterior and posterior to cancer cells may be damaged.

In order to replace this radiation therapy, a non-invasive therapy used to treat cancer with a particle beam such as a proton beam or an ion beam has been introduced. However, in most cases, this non-invasive therapy using a particle beam estimates the position of cancer cells using X-rays and computed tomography (CT) each time direct preceding cancer treatment, and determines the position and direction of the particle beam.

Therefore, the particle beam may be inaccurately delivered onto the cancer cells and thus high levels of energy cannot be used, which may cause an increase in time required for treatment. Besides, the accuracy of position determination on cancer cells may be reduced due to a patient's body functions such as respiration or circulation while the particle beam is being delivered, which may damage healthy cells.

### SUMMARY OF THE INVENTION

An aspect of the present invention provides a minimally invasive particle beam cancer therapy apparatus that can be inserted into the body and deliver a particle beam in proximity to cancer cells generated within the body.

According to an aspect of the present invention, there is provided a provided a minimally invasive particle beam cancer therapy apparatus including: a particle beam delivery system delivering a particle beam onto a diseased part formed inside a therapy subject, the particle beam delivery system being partially inserted into the therapy subject when delivering the particle beam; a medical apparatus body shaped like a pipe having a predetermined length and physically connected to the particle beam delivery system, the medical apparatus being partially inserted into the therapy subject in a longitudinal direction along with the particle beam delivery system being partially inserted into the therapy subject to help the insertion of the particle beam delivery system into the therapy subject; and a control system controlling a driving operation of the particle beam delivery system.

The particle beam delivery system may include: a particle beam generation part generating a particle beam having a predetermined energy; a particle beam delivery part inserted into the therapy subject and delivering the particle beam generated from the particle beam generation part onto the diseased part; and a detection part inserted into the therapy subject and detecting the particle beam delivered onto the diseased part.

The particle beam delivery system may include a particle beam generation part inserted into the therapy subject and generating a particle beam having a predetermined energy; a particle beam delivery part inserted into the therapy subject and delivering the particle beam generated from the particle beam generation part onto the diseased part; and a detection part inserted into the therapy subject and detecting the particle beam delivered onto the diseased part.

The control system may include a storage part storing a predetermined therapy program; and a control part controlling a driving operation of the particle beam delivery system according to the therapy program stored in the storage part and a detection result of the detection part.

The detection part may include an image sensor capturing an image of the diseased part; a dosimeter detecting the amount of the particle beam delivered onto the diseased part from the particle beam delivery part; a gamma camera capturing an image of gamma rays generated when the particle beam collides with the diseased part; and a radiation sensor sensing the amount of radiation of the particle beam.

The control part may include a position control unit correcting a delivery position of the particle beam from the particle beam delivery part according to the detection result of the detection part; and a scan control unit controlling a scanning operation of the particle beam delivery part according to the detection result of the detection part.

The medical apparatus body may have one end thereof physically connected to the detection part and the particle beam delivery part and the other end thereof physically connected to the particle beam generation part, and the particle beam generation part may include a particle beam accelerator generating the particle beam and transmitting the particle beam to the particle beam delivery part through the inside of the medical apparatus body.

The medical apparatus body may be flexible, may have one end thereof physically connected to the detection part and the particle beam delivery part, and have the particle beam generation part therein, wherein the particle beam generation part may include a laser generation unit generating a pulsed laser having a predetermined wavelength; and a particle beam generation unit receiving the pulsed laser from the laser generation unit through a transfer pipe and collecting the received pulsed laser to generate the particle beam.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic view illustrating the configuration of a minimally invasive particle beam cancer therapy apparatus according to an exemplary embodiment of the present invention;

FIG. 2 is a schematic view illustrating an example of a minimally invasive particle beam cancer therapy apparatus according to an exemplary embodiment of the present invention;

FIG. 3A is a schematic view illustrating the configuration of a first embodiment of a minimally invasive particle beam cancer therapy apparatus according to the present invention;

FIG. 3B is a schematic view illustrating the configuration of a second embodiment of a minimally invasive particle beam cancer therapy apparatus according to the present invention; and

FIG. 4 is a view illustrating a scanning operation of a particle beam delivery part used in a minimally invasive particle beam cancer therapy apparatus according to an exemplary embodiment of the present invention.

### DETAILED DESCRIPTION OF THE PREFERRED EMBODIMENT

Exemplary embodiments of the present invention will now be described in detail with reference to the accompanying drawings. The invention may, however, be embodied in many different forms and should not be construed as limited to the embodiments set forth herein. Rather, these embodiments are provided so that this disclosure will be thorough and complete, and will fully convey the scope of the invention to those skilled in the art.

In the drawings, if a portion is considered to unnecessarily divert the gist of the present invention, such portion will be omitted, and the same reference numerals will be used throughout to designate the same or like components.

In addition, unless explicitly described to the contrary, the word “comprise” and variations such as “comprises” or “comprising,” will be understood to imply the inclusion of stated elements but not the exclusion of any other elements.

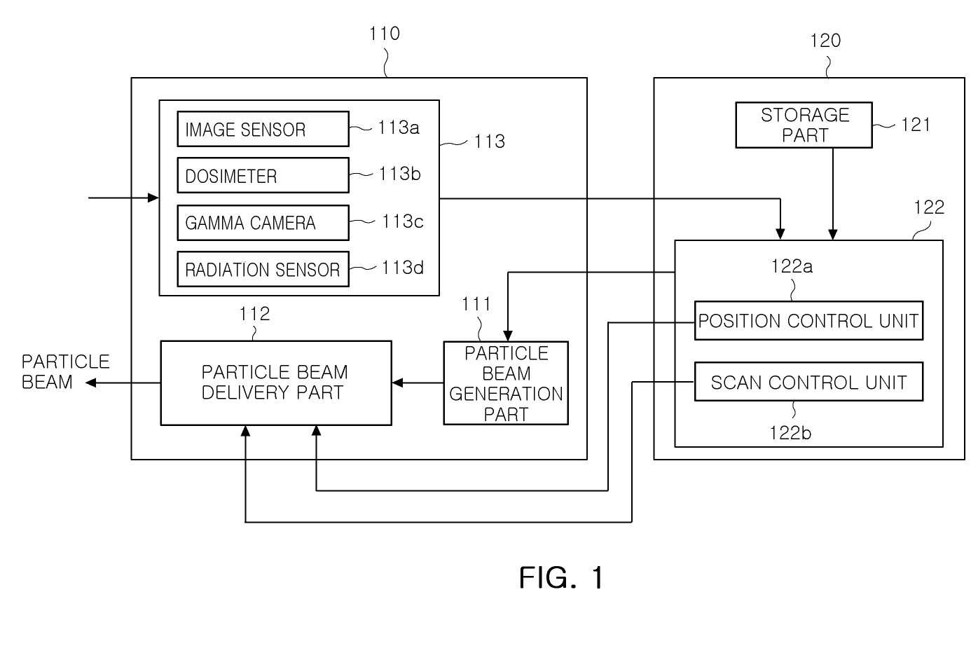

FIG. 1 is a view schematically illustrating the configuration of a minimally invasive particle beam cancer therapy apparatus according to an exemplary embodiment of the invention.

Referring to FIG. 1, a minimally invasive particle beam cancer therapy apparatus 100 according to this embodiment may include a particle beam delivery system 110 and a control system 120.

The particle beam delivery system 110 is at least partially inserted into a therapy subject, more particularly into the human body, and delivers a particle beam, such as a proton beam or an ion beam, onto a diseased part of the body where disease or cancer is developed.

To this end, the particle beam delivery system 110 may include a particle beam generation part 111, a particle beam delivery part 112 and a detection part 113.

The particle beam generation part 111 generates a particle beam having a predetermined level of energy.

When cancer is treated using a particle beam such as a proton beam or an ion beam, the particle beam decreases in kinetic energy while passing through the body by a predetermined distance according to initial kinetic energy, and stops due to the Bragg Peak phenomenon. Therefore, as compared with radiation therapy using photons, the radiation therapy using the particle beam does not damage healthy cells after this stopping distance.

For example, when a proton beam passes through the tissues of the body, the kinetic energy of the proton beam decreases by approximately 10 MeV (mega electron volts) per 1 cm, is absorbed little by little into the body, is reduced in speed and stops around the Bragg peak so that a considerable amount of the proton beam is absorbed into the body.

Therapeutic effects of the proton beam on cancer are known to occur by DNA destruction in tissues caused by kinetic energy of photons and DNA destruction in cells caused by ionization through interaction between kinetic energy and charges of hydrogen ions and atoms and molecules within cells. The Relative Biological Effectiveness (RBE) of the therapy using the proton beam is known to produce similar effects to radiation therapy using X-rays or gamma rays.

When this particle beam is delivered from the outside of the body and is transmitted into the body, the particle beam passes within a predetermined distance (stopping distance), determined by initial kinetic energy, slows down, is stopped and is absorbed into the body, thereby destroying cells. The particle beam loses its kinetic energy while passing through healthy cells and tissues along a path through which the particle beam passes. At this time, the healthy cells and the tissues may be damaged by the particle beam. This damage to the healthy cells may cause secondary cancer development due to cell mutations.

Furthermore, a huge equipment investment needs to be preceded since the particle beam generation part 111 consumes a high amount of energy within the range from 250 to 300 MeV to generate particles to access a location appropriate for treatment of a patient, and radiation and radioactive material generated when accelerating or changing a path need to be shielded. In addition, as for repairs and maintenance, the efficient operation of this huge equipment is limited because of necessary repairs and maintenance workers can gain access to the equipment only after the amounts of radioactivity in the accelerator and attached equipment are reduced.

However, when the particle beam delivery system 110 according to this embodiment is inserted into the body and delivers a particle beam onto a diseased part, damage to healthy cells can be prevented because the particle beam is not transmitted into the body from the outside of the body and consumes a low amount of energy because it consumes a relatively small amount of kinetic energy as compared with the case in which a particle beam is delivered from the outside. Therefore, the size of the particle beam generation part 111 is reduced to thereby facilitate repairs and maintenance.

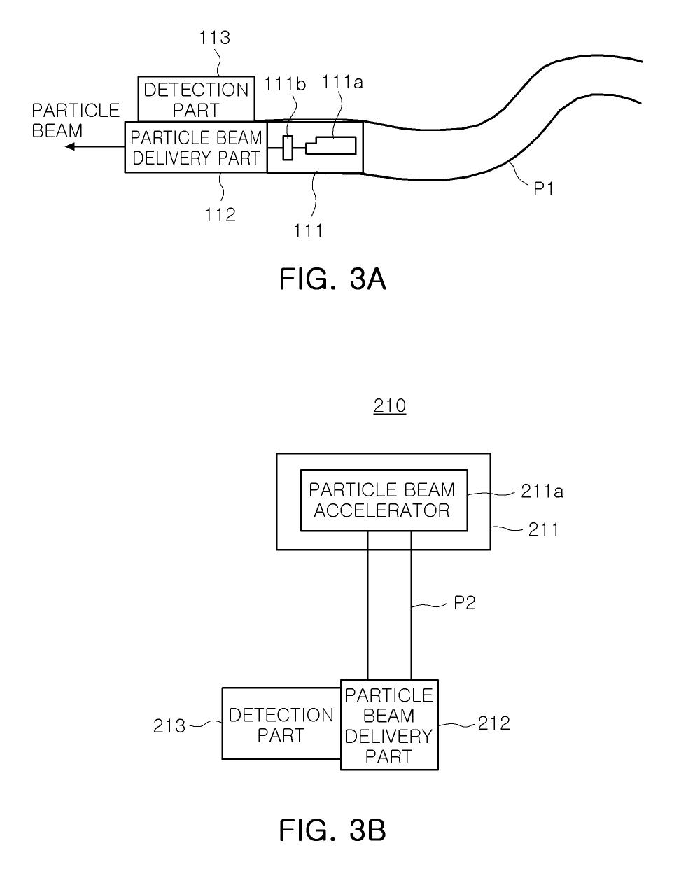

The particle beam generation part 111 may have various configurations according to embodiments in which the particle beam delivery system 110 is inserted into the body. This will be described in detail with reference to FIGS. 3A and 3B.

The particle beam delivery part 112 delivers the particle beam, generated from the particle beam generation part 111, onto the diseased part. At this time, the particle beam delivery part 112 may deliver the particle beam onto the diseased part while the particle beam delivery part 112 is located from the diseased part by a predetermined distance or is as close as possible to the diseased part in order to reduce energy consumption.

The detection part 113 allows the particle beam from the particle beam delivery part 112 to accurately reach the diseased part. To this end, the detection part 113 may include an image sensor 113a, a dosimeter 113b, a gamma camera 113c and a radiation sensor 113d.

The image sensor 113a captures an image of the diseased part inside the body so that the diseased part can be clearly visible to the naked eye. The dosimeter 113b measures the amount of the particle beam, delivered from the particle beam delivery part 112, which makes contact with the diseased part. For example, the dosimeter 113b may measure the amount of particle beam on the basis of the amount of radiation generated when the particle beam makes contact with the diseased part.

The gamma camera 113c captures an image of gamma rays emitted from the diseased part. When the particle beam, delivered from the particle beam delivery part 112, contacts the diseased part, gamma rays are emitted. It is possible to detect which portion of the diseased part the particle beam contacts on the basis of the emitted gamma rays.

The radiation sensor 113d detects the amount of radiation of the particle beam from the particle beam delivery part 112 and controls an energy level of the particle beam according to the detection result, thereby preventing the particle beam from making contact with healthy cells instead of the diseased part.

The control system 120 controls the driving of the particle beam delivery system 110 according to the detection result of the detection part 113.

The control system 120 may include a storage part 121 and a control part 122.

The storage part 121 includes predetermined therapy planning, which may be used to control the driving of the particle beam delivery system 110.

The control part 122 controls the driving of the particle beam generation part 111 and the particle beam delivery part 112 of the particle beam delivery system 110 according to the therapy planning of the storage part 121 and the detection result of the detection part 113. Further, the control part 122 may control the energy level of the particle beam, a particle beam delivery position, and a particle beam delivery amount.

In particular, the control part 122 may include a position control unit 122a and a scan control unit 122b.

The position control unit 122a controls the particle beam delivery part 112 to correct changes in the particle beam delivery position caused by the patient's respiration or circulatory activities so that the particle beam can accurately reach the diseased part. The scan control unit 122b controls the particle beam delivery part 112 so that a uniform amount of the particle beam can be applied over a wide area of the diseased part.

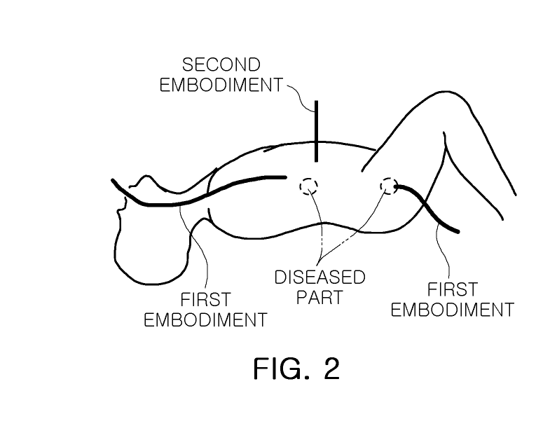

Meanwhile, through not shown, the particle beam delivery system 110, which is used in the medical apparatus according to this embodiment, may be inserted into the body through a medical apparatus body. The medical apparatus body may have various configurations according to embodiments, shown in FIG. 2.

FIG. 2 is a schematic view illustrating a minimally invasive particle beam cancer therapy apparatus according to an exemplary embodiment of the invention.

Referring to FIG. 2, a particle beam delivery system, which is used in the minimally invasive particle beam cancer therapy apparatus according to this embodiment, maybe embodied in various forms according to where a diseased part is generated within the body.

That is, when the diseased part is formed in the internal organs of the body, the particle beam delivery system may be inserted through the mouth or the anus, which is the opening, naturally formed in the body, and thus may gain access to the diseased part like a first embodiment as shown in FIG. 2.

When it is impossible to access the diseased part through the openings naturally formed within the body, an opening may be artificially formed within the body so as to access the diseased part like a second embodiment as shown in FIG. 2.

FIGS. 3A and 3B are schematic configuration views illustrating first and second embodiments of a minimally invasive particle beam cancer therapy apparatus according to an exemplary embodiment of the invention.

Referring to FIG. 3A, the first embodiment 110 of the particle beam delivery system, used in the minimally invasive particle beam cancer therapy apparatus, may include a medical apparatus body P1 having flexibility.

The medical apparatus body P1 may be shaped like a pipe having a predetermined length. The particle beam delivery part 112 and the detection part 113 may be connected to one end of the medical apparatus body P1, and the particle beam generation part 111 may be formed inside the medical apparatus body P1.

Since a path, which is naturally formed within the body, is generally curved, the medical apparatus body P1 may have flexibility like an endoscope in order to access the diseased part. On the other hand, the particle beam generation part 111 may be formed inside the medical apparatus body P1 to efficiently transmit the particle beam to the particle beam delivery part 112.

The particle beam generation part 111 may include a laser generation unit 111a and a particle beam generation unit 111b. The laser generation unit 111a generates a pulsed laser having a predetermined wavelength. The particle beam generation unit 111b receives the pulsed laser from the laser generation unit 111a through a transfer pipe and collects the received pulsed laser to generate a particle beam.

Referring to FIG. 3B, a second embodiment 210 of the particle beam delivery system of the minimally invasive particle beam cancer therapy apparatus according to an exemplary embodiment of the invention may have a particle beam delivery system 210 that is divided on the basis of the medical apparatus body P2. That is, a particle beam delivery part 212 and a detection part 213 are physically connected to one end of the medical apparatus body P2, a particle beam accelerator 211 is physically connected to the other end of the medical apparatus body P2. The particle beam from the particle beam accelerator 211 may be delivered to the particle beam delivery part 212 through the inside of the medical apparatus body P2.

In most cases, cancer is finally diagnosed in such a way that a sample is taken by an invasive biopsy through a sophisticated path, which is artificially formed within the body, and the sample is then subjected to a biopsy to determine if a malignant tumor is present.

As such, after it is determined as a malignant tumor through a sophisticated biopsy, final therapies including surgery, radiation and medication are determined. Here, cancer diagnosis and therapy thereof will be efficiently performed using the second embodiment of the particle beam delivery system according to the invention through the path, having been used for diagnosis directly after a patient is diagnosed with a malignant tumor.

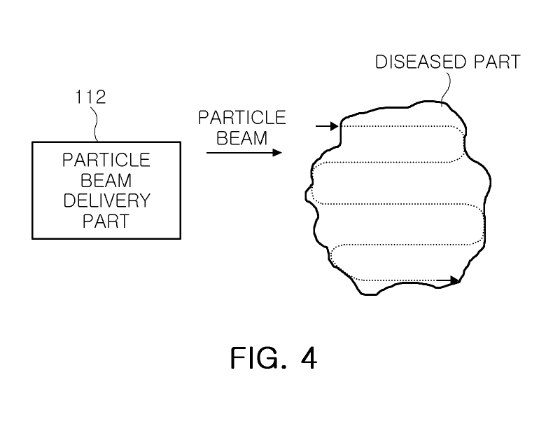

FIG. 4 is a view illustrating a scanning operation of a particle beam delivery part used in a minimally invasive particle beam cancer therapy apparatus according to an exemplary embodiment of the invention.

Referring to FIG. 4, the particle beam delivery part used in the invasive particle beam cancer therapy apparatus can uniformly deliver a particle beam over the diseased part by controlling a particle beam delivery position when the diseased part is wider than particles of the particle beam being delivered.

As described above, according to exemplary embodiments of the invention, the destruction of cancer cells alone can be maximized by scanning a particle beam with a small amount of kinetic energy while the particle beam is delivered in close proximity to a diseased part, especially right next to cancer cells.

Furthermore, a medical apparatus according to an exemplary embodiment of the invention performs a biopsy by checking the position of cancer cells by an endoscope such as the second embodiment, and checks the position of the cancer cells using a detection part in real time to thereby eliminate the uncertainty of the position of the cancer cells and accurately supplies an ion beam to the cancer cells by tracking variations in position of the cancer cells caused by respiration or circulatory activities through position correction. Accordingly, therapeutic effects on cancer can be obtained in a short period of time by delivering a large amount of particle beam onto cancer cells alone while causing little damage to healthy cells by eliminating the uncertainty of the position of the cancer cells, so that patients' pain and inconvenience can be minimized and a more efficient utilization of therapy equipment can be expected.

Furthermore, radiation and radioactive material maybe generated during therapy when all the substances inside the body including healthy cells present along the path through which the particle beam passes collide with particles having high energy, and radiation may be continuously generated from the radioactive material until radioactivity is not detected even after the therapy is finished. However, the medical apparatus according to an exemplary embodiment of the invention uses a particle beam with low kinetic energy to reduce the destruction of the nuclei of the substances inside the body that make contact with the particle beam and reduce the amount of radioactive material being generated since the particle beam is exposed within a small space, thereby significantly reducing indirect radiation exposure to people around a patient as well as the patient.

As set forth above, according to exemplary embodiments of the invention, a minimally invasive particle beam cancer therapy apparatus is inserted into a therapy subject and delivers a particle beam in proximity to a cancer cell generated within a therapy subject to thereby prevent damage to healthy cells, power consumption and equipment size can be reduced with the use of a particle beam having a relatively small amount of energy, and cancer diagnosis and therapy can be performed at the same time by inserting the apparatus into the therapy subject.

Furthermore, a particle beam is focused onto a cancer cell to prevent damage to healthy cells, and therapy time can be reduced since a large amount of particle beam can be delivered as compared with an existing method.

While the present invention has been shown and described in connection with the exemplary embodiments, it will be apparent to those skilled in the art that modifications and variations can be made without departing from the spirit and scope of the invention as defined by the appended claims.

What is claimed is:

1. A minimally invasive particle beam cancer therapy apparatus comprising:
a particle beam delivery system delivering a particle beam onto a diseased part formed inside a therapy subject, the particle beam delivery system being partially inserted into the therapy subject when delivering the particle beam;
a medical apparatus body shaped like a pipe having a predetermined length and physically connected to the particle beam delivery system, the medical apparatus being partially inserted into the therapy subject in a longitudinal direction along with the particle beam delivery system being partially inserted into the therapy subject to help the insertion of the particle beam delivery system into the therapy subject; and
a control system controlling a driving operation of the particle beam delivery system.

2. The minimally invasive particle beam cancer therapy apparatus of claim 1, wherein the particle beam delivery system comprises:
a particle beam generation part generating a particle beam having a predetermined energy;
a particle beam delivery part inserted into the therapy subject and delivering the particle beam generated from the particle beam generation part onto the diseased part; and
a detection part inserted into the therapy subject and detecting the particle beam delivered onto the diseased part.

3. The minimally invasive particle beam cancer therapy apparatus of claim 2, wherein the control system comprises:
a storage part storing a predetermined therapy program; and
a control part controlling a driving operation of the particle beam delivery system according to the therapy program stored in the storage part and a detection result of the detection part.

4. The minimally invasive particle beam cancer therapy apparatus of claim 3, wherein the control part comprises:
a position control unit correcting a delivery position of the particle beam from the particle beam delivery part according to the detection result of the detection part; and
a scan control unit controlling a scanning operation of the particle beam delivery part according to the detection result of the detection part.

5. The minimally invasive particle beam cancer therapy apparatus of claim 2, wherein the detection part comprises:
an image sensor capturing an image of the diseased part;
a dosimeter detecting the amount of the particle beam delivered onto the diseased part from the particle beam delivery part;
a gamma camera capturing an image of gamma rays generated when the particle beam collides with the diseased part; and
a radiation sensor sensing the amount of radiation of the particle beam.

6. The minimally invasive particle beam cancer therapy apparatus of claim 1, wherein the particle beam delivery system comprises:
a particle beam generation part inserted into the therapy subject and generating a particle beam having a predetermined energy;
a particle beam delivery part inserted into the therapy subject and delivering the particle beam generated from the particle beam generation part onto the diseased part; and
a detection part inserted into the therapy subject and detecting the particle beam delivered onto the diseased part.

7. The minimally invasive particle beam cancer therapy apparatus of claim 6, wherein the control system comprises:
a storage part storing a predetermined therapy program; and
a control part controlling a driving operation of the particle beam delivery system according to the therapy program stored in the storage part and a detection result of the detection part.

8. The minimally invasive particle beam cancer therapy apparatus of claim 7, wherein the control part comprises:
a position control unit correcting a delivery position of the particle beam from the particle beam delivery part according to the detection result of the detection part; and
a scan control unit controlling a scanning operation of the particle beam delivery part according to the detection result of the detection part.

9. The minimally invasive particle beam cancer therapy apparatus of claim 6, wherein the detection part comprises:
an image sensor capturing an image of the diseased part;
a dosimeter detecting the amount of the particle beam delivered onto the diseased part from the particle beam delivery part;
a gamma camera capturing an image of gamma rays generated when the particle beam collides with the diseased part; and
a radiation sensor sensing the amount of radiation of the particle beam.

10. The minimally invasive particle beam cancer therapy apparatus of claim 2, wherein the medical apparatus body has one end thereof physically connected to the detection part and the particle beam delivery part and the other end thereof physically connected to the particle beam generation part, and
the particle beam generation part comprises a particle beam accelerator generating the particle beam and transmitting the particle beam to the particle beam delivery part through the inside of the medical apparatus body.

11. The minimally invasive particle beam cancer therapy apparatus of claim 6, wherein the medical apparatus body is flexible, has one end thereof physically connected to the detection part and the particle beam delivery part, and has the particle beam generation part therein,
wherein the particle beam generation part comprises:
a laser generation unit generating a pulsed laser having a predetermined wavelength; and
a particle beam generation unit receiving the pulsed laser from the laser generation unit through a transfer pipe and collecting the received pulsed laser to generate the particle beam.
